(19) United States
(12) Patent Application Publication
(10) Pub. No.: US 2005/0118567 A1
(43) Pub. Date: Jun. 2, 2005

(54) Method for determining sensitivity to a bacteriophage

(76) Inventors: Carl R Merril, Bethesda, MD (US); Sankar Adhya, Gaithersburg, MD (US); Dean M. Scholl, Kensington, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/498,428

(22) PCT Filed: Jan. 23, 2003
(86) PCT No.: PCT/US03/02179

Related U.S. Application Data
(60) Provisional application No. 60/351,458, filed on Jan. 23, 2002.

(51) Int. Cl. C12Q 1/70

(52) U.S. Cl. 435/5

(57) ABSTRACT

This disclosure provides methods of selecting a therapeutic bacteriophage and identifying bacteria in a sample. The sample may be obtained from a plant or animal subject diagnosed with a disease caused by a bacterial infection or an object suspected of being exposed to a bacterium. The activity of reporter molecules, either encoded in the bacteriophage genome or added during sample analysis, is used to determine whether bacteriophages are capable of infecting assayed bacteria. Also provided are methods of selecting a bacteriophage for potential use in treating of bacterial infection, based upon the selectivity of the bacteriophage host range for the bacterium. The bacteriophages or bacteria may be immobilized in an array, such that multiple bacteriophages and/or bacteria may be assayed. Kits are also provided.

### PRIORITY CLAIM

This application claims the benefit of U.S. Provisional Application No. 60/351,458, filed Jan. 23, 2002, which is incorporated by reference in its entirety herein.

### FIELD

The present disclosure relates to methods of selecting bacteriophages capable of infecting bacteria, for example selecting particular bacteriophages therapeutically useful for treating bacterial infections.

### BACKGROUND

Disease-causing bacteria can threaten the health of humans, animals, and plants. Tuberculosis, dysentery, pneumonia, meningitis, cholera, anthrax, Lyme disease, and brucellosis are just a few of the many bacterial diseases that can be harmful to humans and animals. Infectious pathogenic bacteria also cause numerous plant diseases, such as fire blight, crown gall, and citrus canker, affecting a broad range of plants, including grapes, fruit and vegetable crops, tobacco, and nursery stocks.

Traditionally, chemical antibiotics, such as penicillin, streptomycin, and cephalosporin, have been used to treat a variety of bacterial infections. However, bacterial resistance to antibiotics is an increasingly serious problem in human and veterinary health as well as agriculture. Many experts believe that strains of disease-causing bacteria resistant to all common antibiotics (commonly called “superbugs” or multiple drug resistant bacteria) will arise in the next 10 to 20 years. In fact, multidrug-resistant strains of tuberculosis have already been reported in several countries around the world. Yet, despite advances in pharmacology and biotechnology, few new antibiotics have been developed over the past 30 years.

Bacteriophages offer a promising therapeutic alternative to antibiotics, as is demonstrated by the ability of specific bacteriophage to rescue 100% of mice infected with bacteria when administered following bacterial challenge (see Biswas et al., Infection and Immunity 70(1): 204-210, 2002). A bacteriophage is a virus that selectively infects bacteria, such as a particular species or strain of bacteria. For example, a mycobacteriophage is a virus that attacks Mycobacterium species, including Mycobacterium tuberculosis. However, because bacteriophages can be highly host-specific, determining whether a phage would be therapeutically useful against a particular bacterium or strain of bacteria can be a time consuming and labor-intensive process. Even after therapeutically effective bacteriophages are found and determined to be effective against particular species of bacteria, it is still possible that some strains of bacteria are at least partially resistant to treatment with bacteriophages.

To avoid continued progression of a bacterial disease, it is often important to identify specific bacteriophages that are suitable for treating a particular bacterial infection. Since many bacterial pathogens can create a serious infection within a few days, or even a few hours, a need exists for a method of rapidly ascertaining whether a particular strain of bacteria is susceptible to a potentially therapeutic bacteriophage.

### SUMMARY

Disclosed herein are methods for identifying bacteriophage infection of a host bacterium. In one example method, a number of different recombinant bacteriophages are provided, each bacteriophage containing a reporter nucleic acid capable of being expressed when the bacteriophage infects a bacterial cell. These bacteriophages are contacted with a sample contaminated by a bacterium. Expression of the reporter is then detected, such expression indicating that the bacteriophages have infected a bacterial cell. Any appropriate reporter may be used, such as luciferase (SEQ ID NO: 2), green fluorescent protein (including other color derivatives of this protein) (SEQ ID NO: 4), β-galactosidase, or chloramphenicol acetyl transferase (SEQ ID NO: 6).

In another example method, a reporter molecule that is active only upon infection of bacteria by bacteriophage is added to a sample. Activity of the reporter molecule is then detected, such activity indicating that the bacteriophages have infected the bacterial cells. Any appropriate reporter may be used, such as luciferin or luciferase.

In some embodiments, at least two different recombinant bacteriophages are independently selected for use. These bacteriophages may be selected from (without limitation) mycobacteriophage, A511, L5, T4, T7, P58, λ, K5, K1, PM2, P22, K1-5, SP6, twort phage, phi20, T12, H4489a, ENB6, RZh, or IRA. Exemplary recombinant bacteriophages include A511::luxAB, phAE40, PhiV10::luxABcamA1-23 and K1-5::luxAB.

The number of recombinant bacteriophages used to screen a sample may vary according to factors such as the needs of the user, the number of recombinant bacteriophages available, and the number of samples or aliquots to be tested. Particular embodiments of the screening method employ at least two different recombinant bacteriophages, such as 5, 10, 25, 50, 100, 500, 1000, 5000, or more bacteriophages.

The sample screened with a bacteriophage may be obtained from the environment, a plant, or an animal, may be obtained from a culture collection, or may be produced in culture. In some embodiments, the sample is obtained from a plant or animal subject (such as a mammal), or the environment, which is suspected of being infected with or contaminated by bacteria, such as a bacterial biowarfare agent. In other embodiments, a sample is obtained from a plant or animal subject diagnosed with a disease caused by a bacterial infection. The sample may include a cell, tissue, biopsy, secretion or exudate, fluid, or gastric contents obtained from the subject. Examples of samples include, but are not limited to, blood, lymph, urine, a skin scrape or swab, serum, a surface washing, plasma, cerebrospinal fluid, saliva, sputum, stool, vomitus, milk, tears, sweat, or biopsied tissue. In alternative embodiments, the sample is obtained from an environmental locus, such as a food supply, water source, area of soil, or a building.

The sample may be directly contacted with the bacteriophage, or the sample may be processed in some manner before being contacted with the bacteriophage. For example, the sample may be diluted with a solvent, or the number of bacteria may be expanded in culture prior to contact with the bacteriophage. The bacteriophage may be individually compartmentalized, such as within bottles, tubes, or in individual wells of a multi-well plate.

In some embodiments, a treatment is selected for a subject suffering from a bacterial infection. In such embodiments, a sample containing a bacterium is obtained from the subject and contacted with multiple different recombinant bacteriophages, each containing a reporter nucleic acid. Because expression of the reporter indicates that a bacteriophage is capable of infecting the bacteria, one or more bacteriophages expressing the reporter may be selected for treating the subject. A therapeutically effective amount of the selected bacteriophage may then be administered to the subject, as it will be expected that the selected bacteriophage will have a therapeutic effect against the particular pathogen. The bacteriophage administered to the subject may be the same one contacted with the sample (for example, a recombinant bacteriophage containing a reporter nucleic acid), may be a different recombinant bacteriophage based on the same native variant (for example, without the reporter, or which expresses a therapeutic protein), or may be a non-recombinant, native variant of the bacteriophage. In therapeutic embodiments, the administered bacteriophage need not have the reporter nucleic acid, although other non-native nucleic acids can be expressed by the bacteriophage (such as nucleic acids encoding antibacterial peptides).

In other embodiments, the method includes screening bacteriophages capable of infecting bacteria. In such embodiments, plural different recombinant bacteriophages are provided, with each bacteriophage containing a reporter nucleic acid. Each bacteriophage is contacted with a sample that contains a bacterium, and expression of the reporter is detected. A bacteriophage expressing its reporter is thus capable of infecting that species or strain of bacteria.

Kits for the above embodiments are also disclosed. Such kits may include different bacteriophages, directions for contacting each bacteriophage with the sample, and one or more means for detecting the reporter, or devices in which the contacting can be performed. In some embodiments, bacteria known to fall within the host range of a provided bacteriophage may be included as positive controls. In particular embodiments, multi-well plates are provided, such that multiple different bacteria can be placed in different wells and tested by exposure to the same or different bacteriophages. Alternatively, multiple different bacteriophages are placed in different wells and exposed to the same or different bacteria of interest. Such assays permit the rapid identification of potentially therapeutic bacteriophages, or the identification of bacteriophages that are particularly suitable for the infection of a particular pathogen. Arrays of bacteriophages also may be provided for these and other purposes.

### BRIEF DESCRIPTION OF THE DRAWINGS

FIGS. 1A-1D are schematic diagrams that illustrate the construction of a recombinant bacteriophage. FIG. 1A illustrates the entire genome of a generic bacteriophage measuring 20 kb long. FIG. 1B is an enlarged section of the genome of FIG. 1 showing the capsid protein sequence (cps), its associated promoter, a transcription termination (TT) site, and a restriction site. FIG. 1C illustrates insertion of a reporter nucleic acid sequence encoding a luciferase fusion protein (luxAB) after digestion at the restriction site. FIG. 1D shows the recombinant bacteriophage genome segment after insertion of the luxAB nucleic acid. The dotted arrow indicates the region of transcription.

FIGS. 2A-C are schematic diagrams that illustrate production of reporter protein following infection. In FIG. 2A, a bacteriophage is shown infecting a bacterial cell. FIG. 2B illustrates production of viral components early in lytic phase. The separate parts of the bacteriophage capsule and copies of the bacteriophage genome are shown, as well as transcribed reporter proteins indicated by the plus signs (+). FIG. 2C illustrates the end of the lytic phase. Assembled bacteriophages and transcribed reporter proteins are shown being released from a lysed bacterial cell.

FIG. 3 is a schematic drawing of a screening array having a plurality of wells, one of which is illustrated in phantom. This figure also illustrates the screening of a sample contaminated by an unknown bacteria using different recombinant bacteriophages—each specific for a different bacterium—individually separated in the wells of a multi-well plate. While each of the wells contains a different recombinant bacteriophage, only one well is fully illustrated for simplicity. A sample containing the unknown bacteria is aliquotted into each of the wells of the multi-well plate and, following incubation of the plate, a positive reaction (for example, infection of bacteria in the sample by a recombinant bacteriophage) is indicated by the highlighted well. Thus, the positive reaction indicates that the bacteriophage within the well is specific for a bacterium contaminating the sample.

FIG. 4 is a schematic drawing similar to FIG. 3, but which illustrates the selection of a recombinant bacteriophage capable of infecting certain bacteria using different types of bacteria individually separated in the wells of a multi-well plate. While each of the wells contains a different bacterial strain or species, only one well is fully illustrated for simplicity. A sample containing the recombinant bacteriophage is aliquotted into each of the wells of the multi-well plate and, following incubation of the plate, a positive reaction (for example, infection of bacteria in the sample by a recombinant bacteriophage) is indicated by the highlighted wells. Thus, the positive reaction indicates that the bacteria within the wells fall within the host range of the bacteriophage in the sample.

### BRIEF DESCRIPTION OF THE SEQUENCE LISTING

The nucleic and amino acid sequences listed in the accompanying sequence listing are shown using standard letter abbreviations for nucleotide bases, and one-letter code for amino acids, as defined in 37 C.F.R. § 1.822. Only one strand of each nucleic acid sequence is shown, but the complementary strand is understood as included by any reference to the displayed strand. In the accompanying sequence listing:

SEQ ID NO: 1 shows the nucleic acid sequence encoding the Photinus pyralis (firefly) luciferase gene.

SEQ ID NO: 2 is the predicted protein sequence of Photinus pyralis (firefly) luciferase.

SEQ ID NO: 3 shows the nucleic acid sequence encoding the Aequorea victoria green-fluorescent protein.

SEQ ID NO: 4 is the predicted protein sequence of Aequorea victoria green-fluorescent protein.

SEQ ID NO: 5 shows the nucleic acid sequence encoding the Clostridium difficile catD gene for chloramphenicol acetyl transferase.

SEQ ID NO: 6 is the predicted protein sequence of the Clostridium difficile catD chloramphenicol acetyl transferase.

### DETAILED DESCRIPTION

I. Abbreviations

| | |
|---|---|
| ADP | adenosine diphosphate |
| ATP | adenosine triphosphate |
| CAT | chloramphenicol acetyl transferase |
| DNA | deoxyribonucleic acid |
| ELISA | enzyme-linked immunosorbent assay |
| GFP | green fluorescent protein |
| PCR | polymerase chain reaction |
| RNA | ribonucleic acid |

II. Terms and Explanations

The following explanations of terms are provided in order to facilitate review of the embodiments described herein. Explanations of common terms also may be found in Rieger et al., Glossary of Genetics: Classical and Molecular, 5th edition, 25 Springer-Verlag: New York, 1991; Lewin, Genes VII, Oxford University Press: New York, 1999; Dictionary of Bioscience, Mcgraw-Hill: New York, 1997; and Bergey's Manual of Determinative Bacteriology, 9^{th} ed., Williams & Wilkins: Baltimore, 1994.

The singular forms “a,” “an,” and “the” refer to one or more than one, unless 30 the context clearly dictates otherwise. For example, the term “comprising a bacteriophage” includes single or plural bacteriophages and can be considered equivalent to the phrase “comprising at least one bacteriophage.”

As used herein, “comprises” means “includes” such that “comprising A and B” means “including A and B,” without excluding additional elements.

Amplification of bacteria or bacteriophages. The number of bacteria or bacteriophages may be expanded in culture to provide a greater number or density of cells or viral units. For example, bacteria may be amplified by inoculating them into a suitable growth medium, and phages may be amplified by inoculating a suitable bacterial culture, such a culture growing in liquid broth or on solid agar plates.

Amplification of a nucleic acid. A technique that increases the number of copies of a nucleic acid molecule. An example of amplification is the polymerase chain reaction (PCR), in which a sample containing the nucleic acid is contacted with a pair of oligonucleotide primers under conditions that allow for the hybridization of the primers to nucleic acid in the sample. The primers are extended under suitable conditions, dissociated from the template, and then re-annealed, extended, and dissociated to amplify the number of copies of the nucleic acid. The amplification products may be further processed, manipulated, or characterized by electrophoresis, restriction endonuclease cleavage patterns, oligonucleotide hybridization or ligation, nucleic acid sequencing, or other technique of molecular biology. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in European Patent Appl. 320 308; gap filling ligase chain reaction amplification, as disclosed in U.S. Pat. No. 5,427,930; and NASBA™ RNA transcription-free amplification, as disclosed in U.S. Pat. No. 6,025,134.

Animal. A living, multi-cellular, vertebrate organism, including, for example, mammals, birds, reptiles, and fish. The term mammal includes both human and non-human mammals.

Bacteriophage. A virus that selectively infects prokaryotes, such as bacteria. Many bacteriophages are specific to a particular genus or species of host bacteria, such as mycobacteriophage which infects bacteria of the genus Mycobacterium.

The bacteriophage may be a lytic bacteriophage or a lysogenic bacteriophage. A lytic bacteriophage is one that follows the lytic pathway through completion of the lytic cycle, rather than entering the lysogenic pathway. A lytic bacteriophage undergoes viral replication leading to lysis of the host bacterial cell membrane, destruction of the bacterium, and release of progeny bacteriophage particles capable of infecting other bacterial cells. A lysogenic bacteriophage is one capable of entering the lysogenic pathway, in which the bacteriophage becomes a dormant, passive part of the host bacterium's genome through prior to completion of its lytic cycle. In particular embodiments, the bacteriophage is a lytic bacteriophage.

“Bacteriophage” may be shortened to “phage.”

Host range. The host range of a bacteriophage includes those hosts (e.g., bacteria, such as Escherichia coli) in which a bacteriophage may replicate. A mutation in the host range of a bacteriophage may enable or disable the bacteriophage from replicating in a particular host.

Infect. To cause bacteriophage infection of bacteria. A virus “infects” a cell when it injects or transfers its nucleic acid into the bacterial host cell, with the phage nucleic acid existing independently of the host genome. Infection may lead to expression (transcription and translation) of the bacteriophage nucleic acid within the bacterial host cell and continuation of the bacteriophage life cycle. In the case of recombinant bacteriophage, recombinant sequences within the phage genome, such as reporter nucleic acids, may be expressed as well. Transduction, in which the bacteriophage nucleic acid integrates with the host genome following insertion into the bacterial host cell, may be considered a separate event following infection (for example, a bacterial cell that is transduced must first be infected).

Isolated. An “isolated” biological component (such as a nucleic acid, polypeptide, protein, or bacteriophage) has been substantially separated, produced apart from, or purified away from other biological components (for example, other chromosomal and extrachromosomal DNA and RNA, polypeptides, or other types of bacteriophage). Nucleic acids, polypeptides, proteins, and bacteriophages that have been “isolated” may, for example, have been purified by standard purification methods. The term also embraces nucleic acids, polypeptides, proteins, and bacteriophages that are chemically synthesized or prepared by recombinant expression in a host cell. Exemplary methods of synthesis and purification may be found in Sambrook et al., Molecular Cloning: A Laboratory Manual, CSHL, New York, 2001.

An “isolated” bacterium may have been grown apart from other bacteria. For example, bacteria may be grown in selective growth media that select for the growth of a particular bacterium. Additionally, streak plating or successive dilutions of a culture may be used to produce discrete colonies of isolated bacteria.

Nucleic acid. A deoxyribonucleotide or ribonucleotide polymer, in either single or double stranded form, that forms a nucleic acid sequence. Particular nucleic acid sequences disclosed herein encode reporter proteins.

Plant. A living, multicellular organism characterized by the ability to produce food by photosynthesis, thick cell walls containing cellulose, a lack of the power of locomotion, and a relatively open growth pattern, including members of Kingdom Plantae.

Polypeptide. Any chain of amino acid residues linked by peptide bonds, regardless of length or post-translational modification (for example, glycosylation or phosphorylation).

Protein. An organic molecule made up of one or more polypeptides.

Purified. The term purified does not require absolute purity; rather, it is intended as a relative term. A purified molecule or organism is one in which the molecule or organism is more enriched than it is in its natural environment, such as a preparation in which the molecule or organism represents at least 50% of the total content of similar molecules or organisms within the sample. For example, a purified sample of recombinant K1-5 bacteriophage is one in which the recombinant K1-5 bacteriophage represents at least 50% of all bacteriophage within the sample, and a purified sample of Mycobacterium is one in which at least 50% of all bacteria within the sample are from the genus Mycobacterium.

Recombinant. A recombinant bacteriophage is one that contains a nucleic acid sequence that is not naturally occurring or has a sequence that is made by an artificial combination of two otherwise separated segments of sequence. This artificial combination may be accomplished by chemical synthesis or artificial manipulation of isolated segments of nucleic acids, for example, by genetic engineering techniques or the use DNA transposition. Similarly, a recombinant protein is one encoded by a recombinant nucleic acid molecule. The term recombinant bacteriophage includes bacteriophages that have been altered solely by insertion of a nucleic acid, such as by inserting a nucleic acid encoding a reporter protein.

Reporter sequence. A nucleic acid or nucleic acid fragment capable of expressing a reporter molecule. The reporter may contain exogenous nucleic acid sequences in addition to the start and stop codons, such as promoters, enhancers, repressors, or other expression control sequences.

Reporter. A chemical or biochemical signal (e.g., synthesis of a protein or nucleic acid, emission of light, or color change of a solution) capable of being detected or assayed, such as by Northern or Western blots, bioluminescence detection, color production detection, or measurement of fluorescence. Particular reporters are assayable products not normally found in a target bacterium. Proteins generating a reporter signal (“reporter proteins”) include, for instance green fluorescent protein (GFP) (SEQ ID NO: 4), luciferase (SEQ ID NO: 2), β-galactosidase, and chloramphenicol acetyl transferase (CAT) (SEQ ID NO: 6).

In some embodiments, nucleic acids encoding reporter proteins (e.g., luciferase) may be inserted into the genome of bacteriophage disclosed herein such that the reporter is synthesized during replication of the bacteriophage. Following synthesis, the activity of the reporter protein is used to indicate activity of the bacteriophage (e.g., ability to infect a target bacteria).

In other embodiments, reporter proteins (e.g., luciferase) may be externally added and their activity detected following infection of bacterial by bacteriophages (e.g., the biochemical activity of luciferase, which is activated by release of cellular contents of lysed bacteria, is detected by measuring the emission of light from the sample).

The term “subject” includes living organisms capable of being infected by bacteria, such as humans, non-human animals, and plants.

Therapeutic agent. Includes treating agents, prophylactic agents, and replacement agents.

Therapeutically effective amount or effective amount. A quantity sufficient to achieve a desired effect in vitro or in vivo, such as within a subject being treated. For instance, the effective amount of a bacteriophage can be the amount necessary to inhibit bacterial proliferation, measurably neutralize progression of bacterial infection, or reduce the number of bacteria present. In general, this amount will be sufficient to measurably inhibit or reverse the progress of a bacterial infection.

An effective amount may be administered in a single dose, or in several doses, for example daily, during a course of treatment. However, the effective amount may depend on the composition applied or administered, the subject being treated, the severity and type of the affliction, and the manner of administration.

Transduced, transformed, and transfected. A virus or vector “transduces” a cell when it transfers nucleic acid into the cell and that nucleic acid is integrated into the host bacteria's genome. A cell is “transformed” by a nucleic acid transduced into the cell when that nucleic acid becomes stably replicated by the cell, either by incorporation of the nucleic acid into the cellular genome, or by episomal replication. Transfection is the uptake by eukaryotic cells of a nucleic acid from the local environment and may be considered the eukaryotic counterpart to bacterial transformation.

Transposon or transposome. A segment of DNA that can excise and move to a different position in the genome of a single cell, by a method called DNA transposition. A transposon often encodes its own enzyme transposase, which is required for self-excision by the transposon. In some embodiments, a reporter gene is encoded by a transposon, and the transposon is inserted into the bacteriophage genome, to create a recombinant bacteriophage.

III. Reporter

Reporters may optionally be used with the disclosed methods to detect the activity of the bacteriophage discussed herein. In some embodiments, the reporter is inserted into the bacteriophage genome to create recombinant bacteriophage. Any assayable sequence product, such as a nucleic acid or protein, not ordinarily present in the host bacterium in detectable amounts may be encoded by a reporter nucleic acid. Selecting a reporter nucleic acid for any particular embodied recombinant bacteriophage may depend on a variety of factors, such as ease in detecting the reporter, ability to clone the reporter sequence into the bacteriophage, whether the reporter sequence is found in the bacteriophage's host bacteria, rate or amount of expression in the host bacteria, and available methods of detecting the reporter. While expression of a reporter nucleic acid can be determined using Northern blots, Western blots, or antibody detection (for example, ELISA), many reporter sequence products can be more easily detected, such as luminescent reporters, fluorescent reporters, or pigmented reporters. Specific examples of reporters include green fluorescent protein (GFP) (SEQ ID NO: 4), luciferase (SEQ ID NO: 2), β-galactosidase, and chloramphenicol acetyl transferase (CAT) (SEQ ID NO: 6).

Reporter nucleic acids may be independently isolated and cloned, obtained from commercial sources, or synthesized. For example, the firefly (Photinus pyralis) luciferase coding sequence (SEQ ID NO: 1) is available via GenBank Accession No. M15077 and further described in de Wet et al., Mol. Cell. Biol. 7(2):725-37, 1987; the Aequorea victoria green-fluorescent protein coding sequence (SEQ ID NO: 2) is available via GenBank Accession No. M62654 and further described in Prasher et al., Gene 111(2):229-33, 1992; and the coding sequence of a chloramphenicol acetyl transferase sequence from Clostridium difficile (SEQ ID NO: 3) is available via GenBank Accession No. X15100 and further described in Wren et al., Nucleic Acids Res. 17(12):4877, 1989. The sequence for each of these reporters is included in sequence listing herein. Additionally, a number of reporter nucleic acids are available from commercial sources as well. For example, CLONTECH Laboratories, Inc. (Palo Alto, Calif.) offers nucleic acids that encode Living Colors® green and red fluorescent proteins.

In some embodiments, the reporter sequence used with a recombinant bacteriophage is manipulated or processed prior to cloning into the bacteriophage. For example, a small amount of a reporter sequence may be amplified to provide a greater number of individual nucleic acids available for cloning. Additionally, linkers or adapters containing recognition sites for restriction enzymes, spacers, or tags (such as epitope tags) may be added to the ends of the nucleotide, such as by the methods described in Sambrook et al., Molecular Cloning: A Laboratory Manual, CSHL, New York, 1989 and Ausubel et al., Current Protocols in Molecular Biology, Greene Publ. Assoc. and Wiley-Intersciences, 1998. In particular embodiments, a bacteriophage promoter is inserted upstream of the reporter nucleic acid. In other particular embodiments, the number of nucleotides of the reporter nucleic acid is altered by adding or removing nucleotides, such as by inserting a spacer sequence or removing one or more nucleotides from the reporter nucleic acid. Addition or removal of nucleotides may be useful for maintaining the reading frame for the reporter nucleic acid or ensuring that the overall length of the recombinant bacteriophage nucleic acid is suitable for packaging within the viral particle. In still other particular embodiments, the sequence of the reporter nucleic acid is modified to fit the codon preference of the particular bacteriophage or host bacteria. For example (and without limitation), the sequence of the nucleic acid encoding the luciferase fusion protein (luxAB) inserted into the K1-5 bacteriophage, as described in the Examples below, may be modified to fit the codon preference of the host E. coli K1 and/or K5 strains.

In some embodiments, the reporter nucleic acid may be inserted in various positions within the bacteriophage genome to enhance activity of the reporter protein as described in Carriere et al., J. Clin. Microbiol., 35(12): 3232-3239, 1997. Using this method, the expression of the reporter nucleic acid may be modified. In addition, the reporter gene cassette itself may be modified, to add enhancing features such as strong cis-acting native phage promoters that can modulate expression of the reporter gene, as described in Carriere et al., J. Clin. Microbiol., 35(12): 3232-3239, 1997.

In some embodiments, the reporter nucleic acid may be inserted through the use of DNA transposition to effect insertion of a reporter sequence, as discussed in Example 2 below.

Detecting reporter signals, including the activity of a reporter expressed by a reporter sequence carried by a recombinant bacteriophage, depends on the nature of the reporter. Some reporter signals may be detected by simple visual inspection, though detecting reporters may be aided by various systems or apparatuses. For example, the signal of luminescent reporters, such as luciferase (SEQ ID NO: 2), may be detected using a luminescent detector or luminometer, such as the digital luminescence detector manufactured by InterScience, Inc. (Troy, N.Y.) or the Lumat LB 9501/16 tube luminometer manufactured by Berthold Detection Systems (Pforzheim, Germany). Additionally, because luminescent reporters emit light, the luminescent reporter may be exposed to photographic film for an appropriate period of time (such as about a few minutes, hours, or days) and the film developed using standard photographic techniques. Fluorescent reporter signals may be detected using a fluorescence detector, such as the C & L Dye Fluorometer system available from C & L Instruments, Inc. (Hummelstown, Pa.) or the McPherson 749/750 fluorescence detector available from McPherson, Inc. (Chelmsford, Mass.). Pigmented reporter signals may be detected using a spectrophotometer, such as one of the spectrophotometers manufactured by Beckman Coulter, Inc. (Fullerton, Calif.).

In some embodiments, reporters are not inserted into the bacteriophage genome, but may be added externally during analysis (see Example 7) for signal detection. In one specific, non-limiting embodiment, luciferase is added prior to lysis by the bacteriophage. Upon lysis, adenylate kinase is discharged into the reaction milieu, catalyzing conversion of adenosine diphosphate (ADP) to adenosine triphosphate (ATP). The externally added luciferase uses ATP as an energy source, resulting in a signal light that is emitted from only those samples in which ATP was generated, i.e., those samples in which bacteriophage lysed bacteria.

IV. Bacteriophage

In some embodiments, the activity of a single bacteriophage may be evaluated. For example, the ability of that bacteriophage to infect multiple different bacteria can be evaluated to help select a bacteriophage of potential therapeutic usefulness against a particular type of bacterial infection. In other embodiments, at least two different recombinant bacteriophages are assessed for activity against one or more bacteria. For example, multiple different bacteriophages are exposed to a particular type of bacteria (such as a pathogenic strain of pneumococcus) to help select bacteriophages that may be of particular utility in treating pneumococcal pneumonia In some examples, at least 10, at least 15, at least 20, at least 50 at least 100, at least 500, at least 1000, or at least 5000 different bacteriophages are exposed to a pathogen of interest (for example, pneumococcus) to help identify one or more bacteriophages that may be clinically useful.

In some embodiments, the bacteriophages employed may be of a particular type or class. For example, if the bacterial sample is obtained from a human, bacteriophages active against bacteria infecting humans (for example, Mycobacterium, enterococcus, or staphylococcus) may be used. Likewise, if the sample is obtained from a plant, bacteriophages active against bacteria infecting plants (for example, Agrobacterium, Xanthomonas campestris pv. citri, enterococcus, staphylococcus) may be used. Alternatively, combinations of different types or classes can be employed.

While any recombinant bacteriophage may be employed, bacteriophage active against bacteria pathogenic to plants and animals are of particular interest, such as bacteriophage capable of infecting (or transducing) bacteria belonging to the following genera: Escherichia, Shigella, Salmonella, Erwinia, Yersinia, Bacillus, Vibrio, Legionella, Pseudomonas, Neisseria, Bordetella, Helicobacter, Listeria, Agrobacterium, Staphylococcus, Streptococcus, Enterococcus, Clostridium, Corynebacterium, Mycobacterium, Treponema, Borrelia, Francisella and Brucella. Bacteriophages employed also may belong to any of the following virus families: Corticoviridae, Cystoviridae, Inoviridae, Leviviridae, Microviridae, Myoviridae, Podoviridae, Siphoviridae, or Tectiviridae. Particular exemplary bacteriophages include: A511, L5, T4, T7, P58, λ, K5, K1, PM2, P22, K1-5, ENB6, IRA, SP6, twort phage, phi20, T12, RZh, and H4489a; exemplary recombinant bacteriophages include A511::luxAB (Loessner et al., Appl. Environ. Microbiol., 62(4):1133-40, 1996), phAE40 (Riska et al., J. Clin. Microbiol., 35(12):3225-31, 1997), PhiV10::luxABcamA1-23 (Waddell, T. E., and Poppe, C., FEMS Microbiol. Lett. 182:285-89, 2000), and K1-5::luxAB (described in the Examples below).

In certain embodiments, some or all of the bacteriophages have overlapping host ranges—for example, different bacteriophages can infect and replicate on some or all of the same bacteria. In particular embodiments, the bacteriophages have distinct, non-overlapping host ranges-for example, each bacteriophage infects and replicates within one or more distinct species or strains of bacteria.

The number of different recombinant bacteriophages used in any particular embodiment can vary. Bacteriophages may differ by type of phage (for example, the natural variant bacteriophage used to make the corresponding recombinant bacteriophage), the nucleic acid used to encode the reporter protein, or other characteristic. For example, some embodiments employ different recombinant bacteriophages with each bacteriophage representing a different natural variant of bacteriophage. In other embodiments, the bacteriophages differ by type and/or the reporter expressed. Particular embodiments employ 96 different phages, such as embodiments where each well of a 96-well plate contains a different bacteriophage.

Recombinant bacteriophage may be produced using a variety of techniques, such as the techniques described in Sambrook et al., Molecular Cloning: A Laboratory Manual, CSHL, New York, 1989; Ausubel et al., Current Protocols in Molecular Biology, Greene Publ. Assoc. and Wiley-Intersciences, 1998; Loessner et al., Appl. Environ. Microbiol. 62(4):1133-40, 1996; Donnelly-Wu et al., Mol. Microbiol. 7(3):407-17, 1993; Lee et al., Proc. Natl. Acad. Sci. USA 88(8):3111-15, 1991; or Goryshin and Reznikoff, J. Biol. Chem., 273(13):7367-7374, 1998. While techniques for producing particular recombinant bacteriophage may vary according to considerations such as the type and size of the phage, type and size of the reporter sequence, and addition of other sequence elements, many such techniques involve a similar series of steps:
1. Culturing a sufficient amount of bacteriophage particles, such as by plating the bacteriophage on a microbial lawn growing on an agar plate, or growing the bacteriophage in a liquid culture medium.
2. Isolating the bacteriophage particles from the growth medium and host bacteria. Bacteriophage particles may be collected by centrifugation (for example, at 110,000×g induced by 2 hours of centrifugation at 25,000 rpm in a Beckman SW28 rotor) producing a pellet, discarding the supernatant, and resuspending the pellet. Centrifugation methods involving a Cesium choloride or glycerol step gradient also may be used.
3. Extracting the viral genome from the bacteriophage particle. For example, some bacteriophage DNA may be extracted by digesting the viral coat proteins with a protease, such as proteinase K, followed by extraction with phenol:chloroform.
4. Digesting the bacteriophage genome using one or more restriction enzymes to produce bacteriophage arms. In some instances, the bacteriophage arms may be isolated or purified.
5. Introducing the foreign nucleic acid fragments (for example, a reporter sequence) digested in manner to match the restriction sites on the bacteriophage arms.
6. Ligating the bacteriophage arms to the foreign nucleic acid fragment.
7. Reintroducing the recombinant nucleic acid into bacteriophage.
8. Verifying insertion of the foreign nucleic acid into the viral genome. For example, gel electrophoresis may be used to separate recombinant phages from nonrecombinant phages or individual fragments of the viral genome. Insertion of the reporter sequences allows recombinant phages to be identified by expression screening. For example, recombinant bacteriophage could be plated on a bacterial lawn and individual plaques of suitable recombinant bacteriophage identified by detecting the reporter protein expressed by the reporter sequence.

Additional steps may be accomplished when constructing recombinant bacteriophages. For example, nucleic acid sequences, such as virulence sequences, may be disrupted in or removed from the natural variant bacteriophage.

Alternatively, sequences that encode for therapeutic products can be introduced, such as sequences encoding antibacterial peptides. In some embodiments, however, insertion of the reporter sequence into the viral genome may be accomplished without significantly altering the size of the recombinant bacteriophage genome compared to the native variant bacteriophage.

In particular embodiments, a recombinant nucleic acid, such as a reporter sequence or a promoter, is inserted into the bacteriophage genome via homologous recombination, rather than by restriction digestion of the bacteriophage genome followed by insertion and ligation. Briefly, the recombinant nucleic acid is first cloned into a plasmid and flanked by bacteriophage nucleic acid sequences. This recombinant plasmid is then introduced into cells of the bacterial host strain of the bacteriophage via transformation, the bacterial host strain is infected with native type bacteriophage, and the bacteria are plated onto a suitable growth medium. During viral replication, some of the viral genome sequences will undergo homologous recombination with the recombinant plasmid sequences via the flanking bacteriophage sequences within the plasmid. Recombinant bacteriophage can be identified apart from nonrecombinant bacteriophage by assaying plaques within the plated bacteria for reporter activity.

In certain embodiments the recombinant nucleic acid is inserted into the bacteriophage genome using DNA transposition, which involves random transposition of the reporter sequence into the bacteriophage genome using bacterial transposase (see Goryshin and Reznikoff, J. Biol. Chem., 273(13):7367-7374, 1998, and Example 2 below). Briefly, the recombinant nucleic acid (e.g., the reporter nucleic acid sequence) is first cloned into a plasmid and flanked by transposome target nucleic acid sequences. This recombinant segment is then amplified (e.g., by PCR amplification), and mixed with transposase protein (Epicenter Technologies, Madison Wis.). The transposome complex is then introduced into bacterial host cells of the bacteriophage by cell transformation, wherein the bacterial host strain is infected with native type bacteriophage and the bacteria are plated onto a suitable growth medium. During viral replication, some of the viral genome sequences will randomly receive the reporter gene via the flanking transposome elements.

Following transposition, the insertion of the reporter sequence into the recombinant bacteriophage is confirmed by assaying plaques within the plated bacteria for reporter activity as discussed herein.

In certain embodiments, the reporter (for example, luciferase) will not normally be expressed in the bacteria infected by the recombinant bacteriophage and, therefore, will be expressed only if the recombinant bacteriophage infects and replicates in host bacteria. Additionally, in certain embodiments, cloning the reporter sequence to produce the recombinant bacteriophage will be accomplished in a manner that does not alter the host range of the recombinant bacteriophage compared to its natural variant.

In other embodiments, a reporter is externally provided in the reaction milieu (see Schuch et al., Nature, 418: 884-889, 2002). In certain such embodiments, the reporter nucleic acid sequence is not inserted into the bacteriophage genome, but is externally added as a reagent during analysis of the ability of bacteriophage to infect bacteria, as described in Example 7, below.

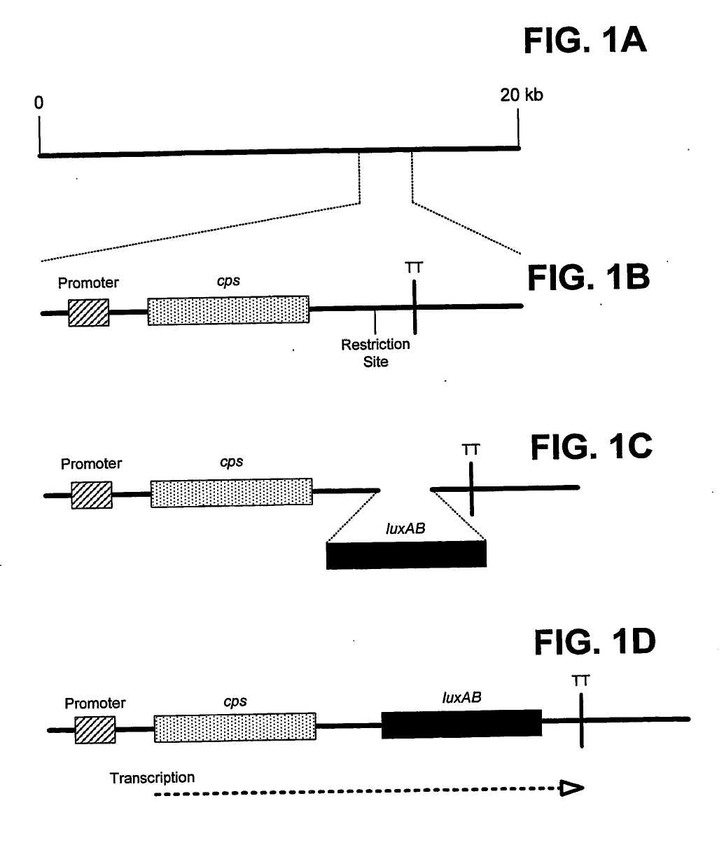

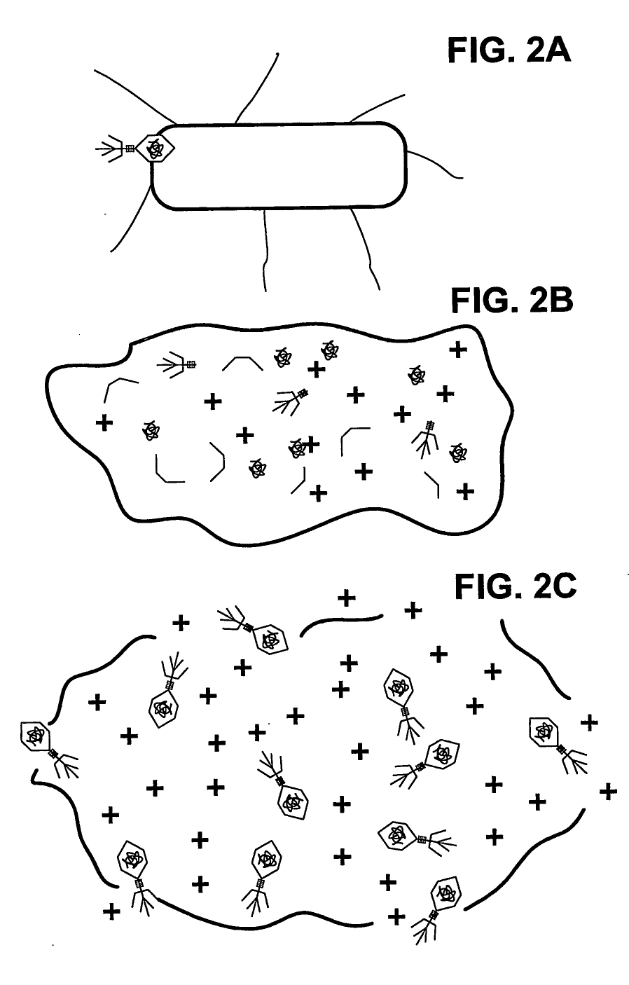

Two or more different recombinant bacteriophages may be used in any particular embodiment, such as at least 10; at least 25, at least 50, at least 100, at least 500, at least 1000, or even at least 5000 different recombinant bacteriophage. In one specific embodiment, 96 different bacteriophages are present, such as a different bacteriophage sample placed in each well of a 96-well plate. In each recombinant bacteriophage, the reporter sequence is under the control of a viral sequence or promoter expressed during or after bacteriophage infection, such as during the lytic phase of the bacteriophage life cycle. For example, as illustrated in FIGS. 1A-D, a recombinant bacteriophage produced by the generalized method above could contain a nucleic acid encoding the luciferase fusion protein (luxAB) inserted into the bacteriophage genome between the 3′ end of a major capsid protein nucleic acid sequence (cps) and the downstream transcription terminator (TT). FIGS. 2A-C illustrate the expression of luciferase from the recombinant bacteriophage during the lytic phase of the bacteriophage life cycle. Luciferase, illustrated by plus signs (+) in FIGS. 2A-C, may be detected before or after it is released from the bacterial cell.

V. Screening and Selecting Bacteriophage

Some embodiments include selecting a bacteriophage for a particular use, such as a therapeutic use, or screening a number of bacteriophages for ability to infect bacteria contained in a sample. In such embodiments, at least two different recombinant bacteriophages (as described above) are contacted with an aliquot of a sample containing a bacterium. In more particular examples, at least 5, 10, 20, 50, 75 or 100 different bacteriophages are contacted with the sample containing the bacteria. Detection of the reporter signal indicates that the recombinant bacteriophage has expressed the reporter and is therefore capable of infecting one or more bacteria contained in the sample. In particular embodiments, the multiple different bacteriophages are immobilized on a substrate (for example, being present in wells of a plate) and are exposed to different aliquots from a single bacterial specimen.

In some embodiments, a sample of purified bacteria is used to assess the efficacy of different bacteriophages in infecting that bacterium. The sample is aliquotted and the aliquots are contacted with different recombinant bacteriophage of the same or different native type. For example (and without limitation), a sample of purified Mycobacterium tuberculosis may be aliquotted onto a multi-well plate. Different bacteriophages may then be introduced into separate wells of the plate, where the different wells contain different recombinant mycobacteriophage, such as recombinant mycobacteriophage of the L5 and D29 native types.

In other embodiments, the sample is obtained from the environment, such as a water or soil sample, a swab of an object, or from a subject, such as an animal or a plant. In particular embodiments, the sample is obtained from a mammal, such as a human or a domesticated animal. In other particular embodiments, the sample is obtained from a nursery plant, an agricultural crop, or a garden.

In some embodiments, a sample is obtained from a subject or the environment to test for the presence of bacteria, for instance as a method to determine the presence of biowarfare agents such as Bacillus anthracis, commonly known as “anthrax.”

In alternative embodiments, a subject is known or suspected to have a bacterial infection, or a sample is obtained from an environment known or suspected to be contaminated by bacteria. In particular therapeutic embodiments (described further below), the subject has been diagnosed with a particular bacterial disease, or is suspected of having a bacterial infection.

The sample can include a cell or tissue taken from the subject, such as a biopsy or extracted gall, or may be obtained from a secretion, exudate, or fluid from the subject. Regarding animal subjects, the sample may be obtained from a bodily fluid (for example, sputum, urine, lymph, blood, cerebrospinal fluid) or gastric contents (for example, the contents of the gastrointestinal system, including stomach contents and stool). The sample in its entirety may be taken solely from the subject, such as by probing or scraping, or may be collected through the addition of some other substance or compound. For example, a sample may simply be blood or urine collected from a subject animal and this blood or urine sample is aliquotted for contacting with the recombinant bacteriophages. As an alternative example, a sample may be obtained by washing the skin of an animal or external surface of a plant with sterile water and collecting the water as it runs off the subject.

Once the sample is collected, it may be directly aliquotted and contacted with the recombinant bacteriophages. Alternatively, in some embodiments, a reporter molecule is added to the sample prior to contact with non-recombinant bacteriophages. In some embodiments, however, the volume of sample obtained may not be sufficient for contacting the recombinant bacteriophages (for example, a sample obtained via a throat swab or skin scrape). In such cases, the sample may be expanded by the addition of a suitable organic or inorganic solvent. Additionally, the number of bacteria may be expanded by culturing in a growth medium to enrich the bacterial density of the sample. In particular examples, the sample is applied to a selective medium, which specifically allows the growth of bacteria of interest. The medium could be, for example, eosin methylene blue (EMB) agar to select Gram-neglected bacteria such as E. coli from urine, or deoxycholate-citrate (DC4) agar to select Salmonella from a stool specimen.

In some embodiments, the recombinant bacteriophages are separated and contacted with an aliquot of the sample to more easily identify which bacteriophage(s) infect the bacteria contained in the sample and express the reporter. In such embodiments, the recombinant bacteriophages are arranged into an “array” of phages placed on an array substrate.

Arrays, as the term is used herein, are arrangements of addressable locations on a substrate, with each address containing a single type of recombinant bacteriophage or a single sample or aliquot of bacteria. A “microarray” is a miniaturized array requiring microscopic examination for detection of the reporter. Larger “macroarrays” allow each address to be recognizable by the naked human eye and, in some embodiments, a reporter signal is detectable without additional magnification. While the following description concerns an array of bacteriophages, it is understood that the same description applies to an inverse arrangement—an array of bacterial samples or aliquots.

The use of the term “array” here is unlike that involved in DNA microchip technology. Rather than having a collection of immobilized target nucleic acids, these arrays contain two or more recombinant bacteriophages physically separated on the array substrate. Additionally, standard DNA microchip technology employs a labeled “target” DNA in solution which hybridizes to one or more “probe” DNAs (identified individual nucleic acid molecules) immobilized on the array. Here, rather than detecting a hybridization, a reporter identifies whether a particular bacteriophage is capable of infecting a known or unknown bacterium contained in a sample or aliquot.

Within an array, each arrayed bacteriophage is addressable—its location may be reliably and consistently determined within the at least the two dimensions of the array surface. Thus, ordered arrays allow assignment of the location of each bacteriophage at the time when it is placed onto the array surface. Usually, an array map or key is provided to correlate each address with the appropriate recombinant bacteriophage. Ordered arrays are often arranged in a symmetrical grid pattern, but bacteriophage could be arranged in other patterns (for example, in radially distributed lines or ordered clusters).

The shape of a bacteriophage address is immaterial. In some embodiments, the bacteriophages are suspended in a liquid medium and contained within square or rectangular wells on the array substrate. However, the bacteriophages may be contained in regions that are essentially triangular, oval, circular, or irregular. The shape of the array itself also is immaterial, though certain embodiments employ a substantially flat substrate that is rectangular or square in shape.

Bacteriophage arrays may vary in structure, composition, and intended functionality. This bacteriophage selection/screening system may employ either a macroarray or a microarray format, or a combination thereof. Such arrays can include, for example, at least 10, at least 25, at least 50, at least 100, at least 500, at least 1000, or more array addresses, usually with a single type of recombinant bacteriophage (or single bacteria sample or aliquot) at each address. Particular arrays employ an ordered number of addresses, such as 12 addresses arranged in three columns and four rows or 96 addresses arranged in eight columns and twelve rows. In the case of macroarrays, sophisticated equipment is usually not required to detect a reporter signal on the array, though quantification may be assisted by known scanning and/or quantification techniques and equipment. Thus, macroarray analysis as described herein can be carried out in most hospitals, agricultural and medial research laboratories, universities, or other institutions without the need for investment in specialized and expensive reading equipment.

Examples of substrates for the bacteriophage arrays disclosed herein include glass (for example, functionalized glass), Si, Ge, GaAs, GaP, SiO₂, SiN₄, modified silicon nitrocellulose, polyvinylidene fluoride, polystyrene, polytetrafluoroethylene, polycarbonate, nylon, fiber, or combinations thereof Array substrates can be stiff and relatively inflexible (for example, glass or a supported membrane) or flexible (such as a polymer membrane). In particular embodiments, a solid substrate is used, such as a multi-well plate. Commercially available multi-well plates suitable for bacteriophage arrays described herein include (but are not limited to) the Microlite line of Microtiter® plates available from Dynex Technologies UK (Middlesex, United Kingdom), such as the Microlite 1+96-well plate, or the 384 Microlite+384-well plate.

Bacteriophages on the array should be discrete, in that reporter signals from an individual bacteriophage address can be distinguished from signals of neighboring bacteriophages, either by the naked eye (macroarrays) or by scanning or reading by a piece of equipment or with the assistance of a microscope (microarrays). Individual bacteriophage particles do not need to be separated, only individual types of recombinant bacteriophages (for example, each well of a plate may contain a liquid suspension of several thousand individual phage particles of a particular recombinant bacteriophage).

Bacteriophage addresses in an array may be a relatively large size, such as large enough to permit detection of a reporter signal without the assistance of a microscope or other equipment. Thus, addresses may be as small as about 0.1 mm across, with a separation of about the same distance. Alternatively, addresses may be about 0.5, 1, 2, 3, 5, 7, or 10 mm across with a separation of a similar or different distance. Larger addresses (larger than 10 mm across) are employed in certain specific embodiments. The overall size of the array is generally correlated with size of the addresses (for example, larger addresses will usually be found on larger arrays, while smaller addresses may be found on smaller arrays). Such a correlation is not necessary, however.

The arrays herein may be described by their densities—the number of addresses in a certain specified surface area. For macroarrays, array density may be about one address per square decimeter (or one address in a 10 cm by 10 cm region of the array substrate) to about 50 addresses per squared centimeter (50 targets within a 1 cm by 1 cm region of the substrate). For microarrays, array density will usually be one address per squared centimeter or more, for instance about 50, about 100, about 200, about 300, about 400, about 500, about 1000, about 1500, about 2,500, or more addresses per square centimeter.

The recombinant bacteriophages may be added to an array substrate in dry or liquid form. Bacterial growth media may be added to the array as well, and the media added to a particular bacteriophage address may be tailored for the bacteria within that particular bacteriophage's host range. For example, growth media suitable for Escherichia may be added to a well containing a recombinant λ bacteriophage in a 96-well plate, while growth media suitable for Salmonella may be added to a well containing a recombinant SP6 bacteriophage. Other compounds or substances may be added to one or more bacteriophage addresses, such as supplemental nutrients, reagents for detecting reporter signal, emulsifying agents, or preservatives.

Particular embodiments include a kit for selecting or screening bacteriophages. Such a kit includes at least two different recombinant bacteriophages (as described above) and instructions. A kit may contain more than two different bacteriophages, such as at least 10, at least 25, at least 50, at least 100, at least 500, at least 1000, at least 5000, or more bacteriophages. The instructions may include directions for obtaining a sample, processing the sample, expanding in culture the bacteria from a sample, and/or contacting each recombinant bacteriophage with an aliquot of the sample. In certain embodiments, the kit includes an apparatus for separating the different bacteriophages, such as individual containers (for example, microtubules) or an array substrate (for example, a 96-well or 384-well microtiter plate). In particular embodiments, the kit includes prepackaged bacteriophages, such as phages suspended in suitable liquid bacterial growth media in individual containers (for example, individually sealed Eppendorfe tubes) or the wells of an array substrate (for example, a 96-well microtiter plate sealed with a protective plastic film). The prepackaged bacteriophages are introduced into different wells of the microtiter plate to prepare the bacteriophage array for “probing” with the bacterial specimen. Kits can also contain instructions for using the components in such a screening test. In alternative embodiments, the kit includes a multi-well plate containing an array of bacteriophages distributed among the wells and premixed with media, reagents, and the like. In such embodiments, a user of the kit can readily aliquot a sample into the wells, incubate the plate for the appropriate time under the correct incubation conditions, and detect any reporter signals produced by the reporter nucleic acid.

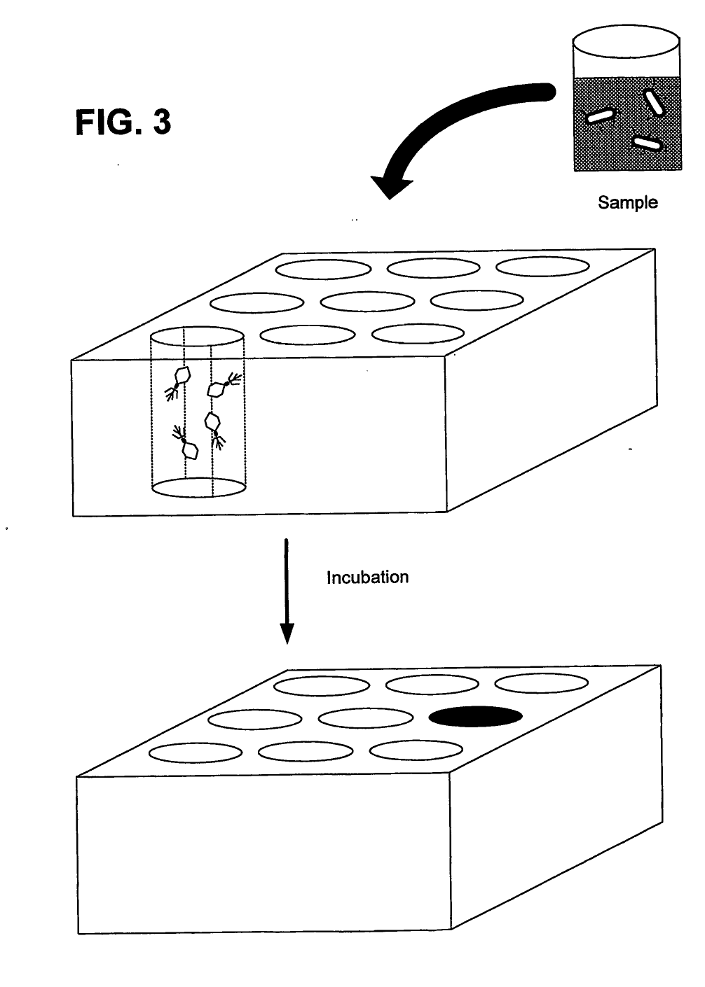

FIG. 3 illustrates one non-limiting embodiment of selecting a bacteriophage. Different types of recombinant bacteriophage are aliquotted into individual wells of a multi-well plate with each well containing a different type of recombinant bacteriophage. As described above, recombinant bacteriophage may differ by native type and/or genomic features, such as the type of reporter nucleic acid inserted into the genome, site of insertion of the reporter nucleic acid, or addition of other genetic elements (for example, promoters, operators, drag-selection markers, etc.). A sample containing an unknown type of bacteria is aliquotted into the wells of plate and the plate is incubated for a time under appropriate conditions. The shaded well indicates a positive result—expression of the reporter nucleic acid following infection of the bacteria by the recombinant bacteriophage contained within that particular well.

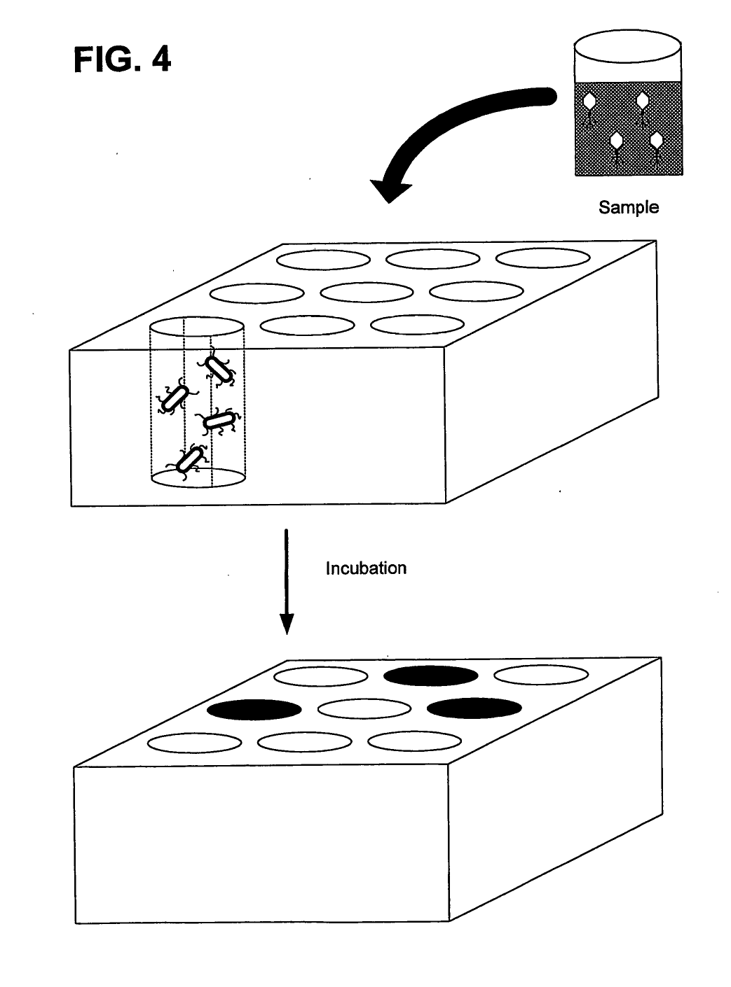

FIG. 4 illustrates an alternative, non-limiting embodiment to that illustrated in FIG. 3. In FIG. 4, each well of the multi-well plate contains a different strain or species of bacteria, while the sample aliquotted into the wells contains a single type of recombinant bacteriophage. Shaded wells indicate a positive result.

VI. Identifying Bacteria

Rather than selecting or screening bacteriophages capable of infecting or transducing bacteria, some embodiments involve identifying or typing particular bacteria based on reporter activity. A sample suspected of containing bacteria may be aliquotted and contacted with bacteriophages, which are known to infect target bacteria, to determine whether the target bacteria are present. In certain embodiments, the bacteria from a sample are isolated and independently cultured prior to contacting with the recombinant bacteriophage. Bacteria in a sample are identified according to whether particular bacteriophages are capable of infecting those bacteria. If a particular bacterium that is infected by a bacteriophage is present, infection will occur and production of the reporter will indicate that this infection has occurred. Presence of the reporter signal therefore indicates the presence of a bacterial strain or species susceptible to infection by the bacteriophage. Additionally, bacteriophage typing of an unknown bacterial species may be accomplished based on the observed sensitivity to an array of recombinant phages.

VII. Therapeutic Uses of Bacteriophage

Selecting or screening bacteriophages may include selecting or screening bacteriophages for therapeutic uses in veterinary, botanical, and human therapeutic applications. In such embodiments, a subject (animal, plant, or human) known or suspected to have a bacterial infection or disease is identified, a sample that would contain bacteria is obtained from that subject, or an isolated bacterial specimen is obtained, and at least two recombinant bacteriophages are contacted with an aliquot of that sample. A recombinant bacteriophage expressing the reporter is selected. In certain embodiments, a bacteriophage of the same type as the selected bacteriophage is administered to the subject. However, the bacteriophage need not have the reporter sequence in it (e.g., the parent bacteriophage from which the recombinant bacteriophage was derived can be administered once the recombinant bacteriophage is selected), although it is possible to administer the bacteriophage containing the reporter sequence in its genome.

Any sample expected to contain bacteria may be obtained from a subject, including a cell, tissue, biopsy, secretion, exudate, or fluid. For example (and without limitation), the sample may be sputum, blood, lymph, sap, urine, a skin scrape or swab, a leaf or root, serum, plasma, cerebrospinal fluid, saliva, a neoplasm, a gall, sputum, a core sample from a stem, stool, vomitus, milk, tears, or sweat. Additionally, as described above, the sample may be processed, or the bacteria in the sample may be expanded or selectively expanded in culture (for example, to select for a particular organism), prior to contacting the recombinant bacteriophages with aliquots of the sample. Alternatively, commercially available bacteria isolates may be used.

Any bacteriophage (including a recombinant bacteriophage) may be used in such therapeutic treatments. However, certain types of bacteriophages may be pre-selected based on considerations such as the type of subject, suspected bacterial infection, and type or quantity of bacteriophage available for administration. For example, if the subject is a person diagnosed with tuberculosis, then recombinant bacteriophages having Mycobacterium within their host ranges may be pre-selected. As another example, if the subject is a pear tree suffering fireblight, then recombinant bacteriophages having Erwinia within their host ranges may be pre-selected. Such pre-selection is not necessary, however.

Once a bacteriophage has been identified for therapeutic administration to the subject, the actual bacteriophage administered to the subject may be the same recombinant bacteriophage that expressed the reporter, or may be a similar bacteriophage, such as a native variant bacteriophage or a different recombinant bacteriophage. For example, if a recombinant K1-5 bacteriophage expressed its reporter when contacted with an aliquot of a sample taken from a dog, then that same recombinant K1-5 bacteriophage may be administered to the dog. Alternatively, a different recombinant K1-5 bacteriophage (for example, one containing a sequence enhancing transcription of the viral genome), or a nonrecombinant native type or mutant K1-5 bacteriophage, may be administered to the dog.

### EXAMPLES

The following examples are provided to illustrate particular features of certain embodiments. The scope of the invention should not be limited to those features exemplified.

### Example 1

### Construction of a Recombinant K1-5 Bacteriophage

This Example discusses how to construct a recombinant,bacteriophage for use with the disclosed methods.

A recombinant K1-5 bacteriophage is constructed by inserting the luxAB DNA sequence into a strongly expressed region of the K1-5 genome downstream of the nucleic acid sequence encoding the capsid protein (cps) via homologous recombination mediated by a recombinant plasmid.

A strong promoter, located upstream of cps, is selectively activated in the course of the expression of the bacteriophage genome following infection, producing many copies of the corresponding mRNA transcripts. Construction of the recombinant K1-5::luxAB bacteriophage is accomplished using a fusion product of the nucleic acid encoding luciferase (luxAB, about 2.1 kbp) from Vibrio harveyi, having suitable translation signals (ribosome binding site, intermediate region, start codon) as described in Loessner et al., Appl. Environ. Microbiol., 62(4): 1133-40, 1996. This fusion product is prepared and inserted into the genome of the K1-5 bacteriophage downstream of cps and before the transcription terminator via the following steps:
a. Construction of the pCK511-k1-5capsid-luxAB plasmid vector. This plasmid is similar to the pCK511-F3s-luxAB plasmid described in Loessner et al., Appl. Environ. Microbiol., 62(4):1133-40, 1996; and U.S. Pat. No. 5,824,468, except that the F3s fragment (a 2123 bp SspI fragment of the A511 bacteriophage) is replaced with a 1000 bp fragment of the K1-5 named “k1-5capsid.” This k1-5capsid fragment corresponds to a region of K1-5 flanking the target site for insertion of the luxAB fragment into the bacteriophage genome located downstream from the cps nucleic acid sequence. The k1-5capsid fragment contains restriction site approximately in its center. Inserting the luxAB fragment at this restriction site provides approximately equal-sized flanking regions of the K1-5 bacteriophage genome available for homologous recombination.
b. Electroransformation of the plasmid vector into an electrocompetent E. coli K1 strain (ATCC strain 23503). The strain is made electrocompetent by growing to an optical density (OD) of 0.8 at 37° C. in LB media, followed by several washes in 15% glycerol. Electrotransformation is accomplished using the Biorad Gene Pulser available from Bio-Rad Laboratories (Hercules, Calif.) according to the manufacturer's instructions.
c. Infection of the transformed E. coli K1 strain with native type K1-5 bacteriophage. After infection of the pCK511-k1-5capsid-luxAB host bacteria, at least a small number of the native K1-5 bacteriophage will undergo homologous recombination with the portions of the k1-5capsid sequence flanking luxAB in the plasmid, thus transferring the luxAB to form recombinant K1-5::luxAB bacteriophage. The transformed bacteria are grown to an OD of 0.4 at 37° C. in LB-ampicillin media. Bacteriophage K1-5 is added at a multiplicity of infection (MOI) of approximately 1 bacteriophage per 10 bacteria, and the OD is monitored until lysis occurs. The lysate is collected by filtering through a 0.45 micron nitrocellulose membrane (available from Millipore Corp., Bedford, Mass.).
d. The lysate is plated and plaqued, using a serial dilution, onto wild type E. coli K1 (ATCC 23503) growing on LB solid agar with 50 μg per ml ampicillin and screened for recombinant K1-5 bacteriophage by assaying plaques for luciferase activity. Recombinant bacteriophages are identified according to the method of Loessner et al., Appl. Environ. Microbiol., 62(4):1133-40, 1996.
e. Confirmation that the luxAB fragment has been inserted at the target site within the recombinant bacteriophage is conducted by sequencing the bacteriophage genome. Sequencing is accomplished by Commonwealth Biotechnologies (Richmond, Va.) using the Sanger chain-termination method.
f. Confirmation of the host range of the recombinant K1-5 bacteriophage by assaying infective activity in E. coli K1. Host range is determined by plaquing the recombinant bacteriophage against a bank of K1 and K5 strains using the method of Scholl et al., J. Virology 75:2509-15, 2001, with minor modifications.

### Example 2

### Use of DNA Transposition to Create Recombinant Bacteriophages

This Example discusses how to create recombinant bacteriophages using DNA transposition to insert the reporter nucleic acid sequence into the bacteriophage genome.

A bacteriophage containing the reporter nucleic acid is constructed using the EZ::TN™ Transposase system as described in Goryshin and Reznikoff, J. Biol. Chem., 273(13):7367-7374, 1998, and commercially available from Epicenter Technologies (Madison, Wis.).

Briefly, the reporter luciferase gene (luxAB) is cloned into the mcs site of the EZ::TN™pMOD-2<mcs> Transposon construction vector. The mcs site is flanked by hyperactive 19 bp mosaic ends (MEs) that are specifically and uniquely recognized by the EZ::TN™Transposase. A transposome is generated either by PCR amplification or restriction enzyme digenstion. The DNA fragment containing the luxAB sequence with the terminal mosaic end elements is incubated with EZ::TN™Transposase in the absence of Mg⁺⁺.

The terminally ME-bound EZ::TN™Transposome is electrotransformed into an electrocompetent E. coli K1 strain (ATCC strain 23503). The strain is made electrocompetent by growing to an optical density (OD) of 0.8 at 37° C. in LB media, followed by several washes in 15% glycerol. Electrotransformation is accomplished using the Biorad Gene Pulser available from Bio-Rad Laboratories (Hercules, Calif.) according to the manufacturer's instructions.

The transformed E. coli K1 strain is infected with native type K1-5 bacteriophage. The transformed bacteria are grown to an OD of 0.4 at 37° C. in LB-ampicillin media. Bacteriophage K1-5 is added at a multiplicity of infection (MOI) of approximately 1 bacteriophage per 10 bacteria, and the OD is monitored until lysis occurs. After infection of the transposome-electrotransformed host bacteria, at least a small number of the native K1-5 bacteriophage will receive the reporter gene by random transposition at an innocuous position that does not affect the plaque-forming ability of the phage. The lysate is collected by filtering through a 0.45 micron nitrocellulose membrane (available from Millipore Corp., Bedford, Mass.).

The lysate is plated and plaqued, using a serial dilution, onto wild type E. coli K1 (ATCC 23503) growing on LB solid agar with 50 μg per ml ampicillin and screened for recombinant K1-5 bacteriophage by assaying plaques for luciferase activity. Recombinants are identified according to the method of Loessner, et al. (1996) or it may be confirmed that the luxAB fragment has been inserted at a non-innocuous region of the bacteriophage genome by restriction mapping and DNA sequencing. Sequencing is accomplished by Commonwealth Biotechnologies (Richmond, Va.) using the Sanger chain-termination method.

The host range of the recombinant K1-5 bacteriophage may be confirmed by assaying infective activity in E. coli K1. Host range is determined by plaquing the recombinant bacteriophage against a bank of K1 and K5 strains using the method of Scholl et al., J. Virology 75:2509-15 (2001) with minor modifications.

### Example 3

### Determination of Optimal Conditions for Luciferase Expression in K1-5::luxAB

This Example discusses how to optimize expression of a reporter gene, such as luciferase, in a recombinant bacteriophage.

The optimal conditions for luciferase expression in a K1-5::luxAB bacteriophage (see Examples #1 and #2) are determined according to the protocol described in Loessner et al., Appl. Environ. Microbiol., 62(4):1133-40, 1996, with slight modification.

Kinetics of light-emitting reaction. K1-5::luxAB infected E. coli K1 cells in LB medium are incubated for 2 hours at 37° C. and placed in a Lumat LB 9506 tube luminometer (Berthold Australia Pty Ltd., Bundoora, VIC, Australia). Emission is detected for a 30 second period following injection of the substrate. Reaction kinetics are determined from a histogram analysis with 20 data points.

Multiplicity of infection. One hundred ill portions of host cells are added to 1 ml bacteriophage suspensions containing 5×10⁷, 5×10⁸, 2.5×10⁸, 5×10⁸, 5×10⁹, and 2.5×10⁹ PFU/ml. Resulting mixtures are incubated and assayed in the same manner, and the signal integration time is 5 seconds.

### Example 4

### Determination of Detection Limits

This Example discusses how to determine the limits of detection for bacteriophage in a sample.

The limits for detecting E. coli K1 and K5 cells in a sample using K1-5::luxAB are determined.

Log-phase cultures of E. coli K1 and K5 strains are diluted with LB medium to low cell densities of 1×10², 5×10², 1×10³, 2.5×10³, 5×10³, and 1×10⁴ CFU/ml. A sample of each dilution culture is infected with 3×10⁸ K1-5::luxAB bacteriophage per ml, incubated for 2 hours at 37° C., and assayed in triplicate for light emission (with an integration time of 5 seconds). Negative controls (no bacteriophage added to the sample) are used to determine background light readings.

### Example 5

### Screening a Sample Obtained from a Subject

This Example discusses how to screen samples obtained from a subject using recombinant bacteriophage.

Identification of bacteria infecting a subject exhibiting symptoms of such an infection is accomplished using recombinant bacteriophage.

A subject (e.g., a human patient) exhibiting symptoms of bacterial infection (for example, fever, headache, abdominal pain, and nausea) is identified, and the following samples are collected from the subject: a 0.01 ml cerebrospinal fluid (CSF) sample, a 1.0 ml sputum sample, and a 1.0 ml blood sample. Each sample is diluted with 4.0 ml of LB broth, thus promoting growth of all bacteria present in the respective sample, and is incubated at 37° C. for 4 hours. After incubation, each sample is distributed by 100 μl aliquots into 30 wells of a 96-well plate. Aliquots of the blood sample are added to wells 1-30, aliquots of the CSF sample are added to wells, 31-60, aliquots of the sputum sample are added to wells 61-90, wells 91-93 serve as positive controls, and wells 94-96 serve as negative controls.

The following five recombinant bacteriophage are obtained: K1-5::luxAB bacteriophage, which infects E. coli K1 bacteria; EBN6:: luxAB bacteriophage, which infects enterococcus bacteria; Twort::luxAB bacteriophage, which infects staphylococcus bacteria; Sp6::luxAB bacteriophage, which infects Salmonella bacteria; and RZh::luxAB bacteriophage, which infects streptococcus bacteria. The bacteria may be obtained from another source or produced using genetic engineering techniques, including the protocol described in Example #1.

Recombinant bacteriophage suspension equivalent to about 3×10⁸ phages/ml is added to six individual wells of the groups of 30 wells corresponding to each of the three samples collected from the patient. For example, the K1-5::luxAB bacteriophage is added to wells 1-6 (blood), 31-36 (CSF), and 61-66 (sputum); while EBN6::1luxAB bacteriophage is added to wells 7-12 (blood), 3742 (CSF), and 67-72 (sputum). This distribution of collected samples and recombinant bacteriophage within the array of 96 wells is summarized in Table 1 (with the 6 control wells not shown).

TABLE 1
Array of Patient Samples and Recombinant Bacteriophage

| | Sample Obtained | | |
|---|---|---|---|
| | from the Patient | | |
| | Blood | CSF | Sputum |
| K1-5::luxAB | 1. | 31. | 61. |
| | 2. | 32. | 62. |
| | 3. | 33. | 63. |
| | 4. | 34. | 64. |
| | 5. | 35. | 65. |
| | 6. | 36. | 66. |
| EBN6:: luxAB | 7. | 37. | 67. |
| | 8. | 38. | 68. |
| | 9. | 39. | 69. |
| | 10. | 40. | 70. |
| | 11. | 41. | 71. |
| | 12. | 42. | 72. |
| Twort::luxAB | 13. | 43. | 73. |
| | 14. | 44. | 74. |
| | 15. | 45. | 75. |
| | 16. | 46. | 76. |
| | 17. | 47. | 77. |
| | 18. | 48. | 78. |
| Sp6::luxAB | 19. | 49. | 79. |
| | 20. | 50. | 80. |
| | 21. | 51. | 81. |
| | 22. | 52. | 82. |
| | 23. | 53. | 83. |
| | 24. | 54. | 84. |
| RZh::luxAB | 25. | 55. | 85. |
| | 26. | 56. | 86. |
| | 27. | 57. | 87. |
| | 28. | 58. | 88. |
| | 29. | 59. | 89. |
| | 30. | 60. | 90. |

Luciferase activity is observed in wells 7-12, thus indicating the subject has an enterococcus infection of the blood.

### Example 6

### Screening a Sample Obtained from a Human Patient

This Example discusses how to screen samples obtained from a human patient for meningitis using recombinant bacteriophage.

The confirmation of E. coli K1 bacteria infection in a human patient having meningitis is accomplished using a recombinant K1-5 bacteriophage prepared according to the protocol described in Example #1.

A patient exhibiting signs of meningitis is identified, and a 0.01 ml cerebrospinal fluid (CSF) sample is collected from the patient. The sample is diluted with 1.0 ml of LB broth, thus promoting growth of all bacteria present in the CSF sample, and is incubated (e.g., at 37° C. for 12 hours). After incubation, the sample is distributed by 100 μI aliquots into each well of a 12-well plate.

A recombinant K1-5::luxAB bacteriophage, as described in Example #1, is used to screen for the presence of E. coli K1 bacteria. K1-5::luxAB bacteriophage suspension equal to about 3×10⁸ phages/ml of the recombinant bacteriophage is added to 8 of the wells of the plate, with the remaining 4 wells serving as positive and negative controls.

Detection of the reporter signal is accomplished by exposing the plate to photographic film for three hours and developing the film. Positive results indicate that the patient has a meningitis infection caused by E. coli K1.

### Example 7

### External Addition of Luciferase to Detect Bacteriophage Infection of Cells

This Example discusses how to use specialized reagents to detect bacteriophage infection of cells without the need for recombinant insertion of a reporter gene into the bacteriophage genome.

Samples from a subject in which the presence of bacteria is being screened are prepared as described herein or as described in Carriere et al., J. Clin. Microbiol., 35(12): 3232-3239, 1997. Cells containing bacteria are grown to a stationary phase (e.g., an optical density of 1.8 to 2.0 at 600 nm), washed and resuspended in bacterial broth (e.g., Middlebrook 7H9 broth, Difco). The samples are incubated (e.g., standing for 24 hours at 37° C.) to optimize their infectibility by bacteriophages. After incubated, bacteriophages are added at the desired multiplicity of infection, for instance 1×10³.

At desired intervals, for instance from 1 to 24 hours after infection, aliquots for analysis are removed, and luciferin (e.g., D-luciferin, Sigma) or a luciferase reagent (e.g., the luciferase reagent provided in Roche Molecular Biochemicals ATP Bioluminescence Assay Kit CLS II or the Promega CellTitle-Glo™ Luminescent Cell Viability Assay) is added to the incubation mixture. Lysis of bacterial strains by the bacteriophage will result in the discharge of adenylate kinase, which can convert adenosine diphosphate (ADP) in the reaction milieu to adenosine triphosphate (ATP). The luciferin/luciferase reagent utilizes ATP for emission of light, enabling the user to detect the bacteria-infecting bacteriophage by identifying wells emitting light at approximately 562 nm. Light emitted from samples may be measured on a luminometer (Perstorp Analytical, Silver Spring Md.), or the samples may be exposed to radiographic film. Those samples that emit light are determined to contain bacteria within the host range of the added bacteriophage.

### Example 8

### Screening a Sample for the Presence of Biowarfare Bacterial Agents

This Example discusses how to screen a sample for the presence of bacterial agents used in biowarfare, such as Bacillus anthracis, commonly known as “anthrax.”

The confirmation of E. coli K1 bacteria infection in a human patient having meningitis is accomplished using a recombinant K1-5 bacteriophage prepared according to the homologous recombination protocol described in Example 1 or DNA transposition as described in Example 2.

A sample is collected from a subject or an object suspected to be contaminated with a bacterial biowarfare agent. The sample is cultured in LB broth, thus promoting growth of all bacteria present in the CSF sample, and is incubated (e.g., at 37° C. for 12 hours). After incubation, the sample is distributed by 100 μl aliquots into each well of a 12-well plate.

A recombinant K1-5::luxAB bacteriophage, as described in Example 1 or Example 2, is used to screen for the presence of E. coli K1 bacteria. K1-5::luxAB bacteriophage suspension equal to about 3×10⁸ phages/ml of the recombinant bacteriophage is added to 8 of the wells of the plate, with the remaining 4 wells serving as positive and negative controls.

Detection of the reporter signal is accomplished by exposing the plate to photographic film for three hours and developing the film. Positive results indicate that the subject or the object is contaminated with bacteria caused by E. coli K1.

Having illustrated and described the principles of the invention by several embodiments, it should be apparent that those embodiments may be modified in arrangement and detail without departing from the principles of the invention. Thus, the invention as claimed includes all such embodiments and variations thereof, and their equivalents, as come within the true spirit and scope of the claims stated below.

1. A method for selecting a bacteriophage capable of infecting a target bacterium, comprising:
contacting at least two different bacteriophages with a sample from a subject comprising the target bacterium in the presence of a reporter capable of generating a detectable signal in response to a lytic activity of a bacteriophage; and
detecting whether the signal is generated, the detection of the signal indicating that the bacteriophage is capable of infecting the target bacterium.

2. The method of claim 1, wherein each bacteriophage comprises a nucleic acid encoding the reporter, wherein the reporter is expressed upon infection of the target bacterium by the bacteriophage such that the activity of the reporter is detected.

3. The method of claim 1, wherein the reporter is added to the sample prior to detection of the reporter signal.

4. The method of claim 1 wherein the at least two different bacteriophages are selected from the group consisting of: mycobacteriophage, K1, K5, K1-5, SP6, T4, T7, ENB6, A511, L5, and IRA.

5. The method of claim 1 wherein the reporter is luciferase, green fluorescent protein, P-galactosidase, or chloramphenicol acetyl transferase.

6. The method of claim 1 wherein the target bacterium comprises a bacterium of the genus Escherichia, Shigella, Salmonella, Erwinia, Yersinia, Bacillus, Vibrio, Legionella, Pseudomonas, Neisseria, Bordetella, Helicobacter, Listeria, Agrobacterium, Staphylococcus, Streptococcus, Enterococcus, Clostridium, Corynebacterium, Mycobacterium, Treponema, Borrelia, Francisella, or Brucella.

7. The method of claim 1, wherein contacting at least two different bacteriophages comprises contacting the sample with at least 10 different bacteriophages.

8. The method of claim 1, wherein contacting at least two different bacteriophages comprises contacting the sample with at least 25 different bacteriophages.

9. The method of claim 1, wherein contacting at least two different bacteriophages comprises contacting the sample with at least 50 different bacteriophages.

10. The method of claim 1, wherein contacting at least two different bacteriophages comprises contacting the sample with at least 100 different bacteriophages.

11. The method of claim 1, wherein contacting at least two different bacteriophages comprises contacting the sample with at least 500 different bacteriophages.

12. The method of claim 1, wherein the sample is obtained from a subject.

13. The method of claim 12 wherein the sample comprises a cell, tissue, secretion or exudate, fluid, gastric contents, blood, lymph, urine, a skin scrape or swab, serum, plasma, cerebrospinal fluid, saliva, sputum, stool, vomitus, milk, tears, or sweat.

14. The method of claim 12, wherein the subject is a mammal.

15. The method of claim 14, wherein the mammal is a human.

16. The method of claim 12, wherein the subject is suspected of having a bacterial infection.

17. The method of claim 12, wherein the subject has been diagnosed with a disease caused by a bacterial infection.

18. The method of claim 1, wherein the sample is obtained from an object suspected to be contaminated with a biowarfare agent.

19. The method of claim 18, wherein the sample is obtained by swabbing the object or collecting liquid wash applied to the object.

20. The method of claim 1 wherein the different bacteriophages are contained in separate wells of a multi-well plate.

21. The method of claim 1 wherein the number of bacteria in the sample is expanded in culture prior to selection.

22. The method of claim 16, wherein the bacteriophage capable of infecting the target bacterium is selected for use in treating the subject's bacterial infection.

23. The method of claim 22, further comprising administering to the subject a therapeutically effective amount of a bacteriophage of the same type as the selected bacteriophage.

24. The method of claim 23, wherein the bacteriophage administered to the subject is a recombinant bacteriophage.

25. The method of claim 23, wherein the bacteriophage administered to the subject is a parent bacteriophage of the selected recombinant bacteriophage.

26. The method of claim 23, wherein the bacteriophage administered to the subject is a native variant of the selected recombinant bacteriophage.

27. The method of claim 23, wherein the subject is a plant.

28. The method of claim 23, wherein the subject is a mammal.

29. The method of claim 28, wherein the mammal is a human.

30. A kit for selecting a bacteriophage capable of infecting a target bacterium, comprising:
at least two different bacteriophages;
a reporter capable of generating a detectable signal in response to an activity of the bacteriophages; and
instructions for contacting each the bacteriophages with a sample and for detecting the signal.

31. The kit of claim 30, wherein each bacteriophage comprises a nucleic acid encoding the reporter, wherein the reporter is expressed upon infection of the target bacterium by the bacteriophage such that the activity of the reporter is detected.

32. The kit of claim 31 further comprising means for detecting expression of the reporter.

33. The kit of claim 30, wherein the reporter is added to the sample prior to detection of the reporter signal.

34. The kit of claim 30, further comprising a substrate upon which the different bacteriophages can be separated and contacted with the sample.

35. The kit of claim 30 wherein the kit comprises at least 24 different bacteriophages.

36. The kit of claim 30 wherein the kit comprises at least 48 different bacteriophages.

37. The kit of claim 30 wherein the kit comprises at least 96 different bacteriophages.

38. The kit of claim 30 wherein the kit comprises an array of bacteriophages at addressable locations in the array.

39. The kit of claim 30, comprising one or more bacteria known to fall within the host range of a provided bacteriophage as a positive control.

40. A method of identifying a bacterium that falls within the host range of a bacteriophage, comprising:
separately contacting at least two different recombinant bacteriophages with a sample comprising a bacterium, where each bacteriophage comprises a nucleic acid encoding a reporter capable of being expressed when the bacteriophage infects a bacterial host cell that is characteristically infected by the bacteriophage; and
detecting whether the reporter is expressed, where expression of the reporter indicates that the bacterium falls within the host range of the bacteriophage.

41. The method of claim 40, comprising more than two different recombinant bacteriophages.

42. The method of claim 41 wherein the at least two different recombinant bacteriophages have overlapping host ranges.

43. The method of claim 42, comprising at least 25 different recombinant bacteriophages.

44. The method of claim 43, comprising at least 50 different recombinant bacteriophages.

45. The method of claim 44, comprising at least 100 different recombinant bacteriophages.

46. The method of claim 45, comprising at least 500 different recombinant bacteriophages.

47. The method of claim 40 where each recombinant bacteriophage is individually isolated.

48. The method of claim 47 where each recombinant bacteriophage is contained in a separate well of a multi-well plate.

49. A method of treating a bacterial infection in a host, comprising:
obtaining bacterial pathogen cells from a host;
contacting different bacterial pathogen cells with different recombinant bacteriophages, where each recombinant bacteriophage comprises a nucleic acid encoding a reporter capable of being expressed when the recombinant bacteriophage infects a bacterial host cell within its host range;
selecting a bacteriophage that expresses the reporter, where expression of the reporter indicates that the selected bacteriophage is capable of infecting a bacterial host cell obtained from the host; and
administering a therapeutically effective amount of the selected bacteriophage to the host, thereby treating the bacterial infection in the host.

50. The method of claim 49 where the host is an animal or a plant.

51. The method of claim 50 where the host is a mammal.

52. The method of claim 51 where the mammal is a human.

53. The method of claim 49 wherein obtaining a bacterial pathogen cell from the host comprises obtaining a bacterial pathogen cell of the genus Escherichia, Shigella, Salmonella, Erwinia, Yersinia, Bacillus, Vibrio, Legionella, Pseudomonas, Neisseria, Bordetella, Helicobacter, Listeria, Agrobacterium, Staphylococcus, Streptococcus, Enterococcus, Clostridium, Corynebacterium, Mycobacterium, Treponema, Borrelia, Francisella, or Brucella.

54. The method of claim 49 wherein administering a therapeutically effective amount of the selected bacteriophage to the host comprises administering a bacteriophage of the same native type as the selected bacteriophage.

55. A device for selecting bacteriophages that are capable of infecting target bacteria, the device comprising:
an array of recombinant bacteriophages at addressable locations in the array, wherein the bacteriophages each include a signal sequence that provides a detectable signal when a bacteriophage infects a bacterium.

56. The device of claim 49, further comprising bacteria in at least some of the addressable locations of the array.

57. The device of claim 55, wherein at least some of the different addressable locations of the array contain different bacteriophage types that are capable of selectively infecting different bacterial strains or species, such that the signal indicates the ability of the bacteriophage to infect the bacterial strain or species that is located at the address from which the signal is provided.


SEQUENCE LISTING (6 sequences)

SEQ ID NO: 1 - DNA, 2387 bp, Photinus pyralis
ctgcagaaataactaggtactaagcccgtttgtgaaaagtggccaaacccataaatttggcaattacaataaagaagctaaaattgtggtcaaactcacaaacatttttattatatacattttagtagctgatgcttataaaagcaatatttaaatcgtaaacaacaaataaaataaaatttaaacgatgtgattaagagccaaaggtcctctagaaaaaggtatttaagcaacggaattcctttgtgttacattcttgaatgtcgctcgcagtgacattagcattccggtactgttggtaaaatggaagacgccaaaaacataaagaaaggcccggcgccattctatcctctagaggatggaaccgctggagagcaactgcataaggctatgaagagatacgccctggttcctggaacaattgcttttgtgagtatttctgtctgatttctttcgagttaacgaaatgttcttatgtttctttagacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtaagcaccctcgccatcagaccaaagggaatgacgtatttaatttttaaggtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccaggtatgtcgtataacaagagattaagtaatgttgctacacacattgtagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacggtgagttaagcgcattgctagtatttcaaggctctaaaacggcgcgtagcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtaatgaagatttttacatgcacacacgctacaatacctgtaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtaaatgaattcgttttacgttactcgtactacaattcttttcataggtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaaatgtaactgtattcagcgatgacgaaattcttagctattgtaatattatatgcaaattgatgaatggtaattttgtaattgtgggtcactgtactattttaacgaataataaaatcaggtataggtaactaaaaa

SEQ ID NO: 2 - protein, 1536 aa, Photinus pyralis
METGLASPALALYSASNILELYSLYSGLYPRALAPRPHETYRPRLEGLASPGLYTHRALAGLYGLGLNLEHISLYSALAMETLYSARGTYRALALEVALPRGLYTHRILEALAPHETHRASPALAHISILEGLVALASNILETHRTYRALAGLTYRPHEGLMETSERVALARGLEALAGLALAMETLYSARGTYRGLYLEASNTHRASNHISARGILEVALVALCYSSERGLASNSERLEGLNPHEPHEMETPRVALLEGLYALALEPHEILEGLYVALALAVALALAPRALAASNASPILETYRASNGLARGGLLELEASNSERMETASNILESERGLNPRTHRVALVALPHEVALSERLYSLYSGLYLEGLNLYSILELEASNVALGLNLYSLYSLEPRILEILEGLNLYSILEILEILEMETASPSERLYSTHRASPTYRGLNGLYPHEGLNSERMETTYRTHRPHEVALTHRSERHISLEPRPRGLYPHEASNGLTYRASPPHEVALPRGLSERPHEASPARGASPLYSTHRILEALALEILEMETASNSERSERGLYSERTHRGLYLEPRLYSGLYVALALALEPRHISARGTHRALACYSVALARGPHESERHISALAARGASPPRILEPHEGLYASNGLNILEILEPRASPTHRALAILELESERVALVALPRPHEHISHISGLYPHEGLYMETPHETHRTHRLEGLYTYRLEILECYSGLYPHEARGVALVALLEMETTYRARGPHEGLGLGLLEPHELEARGSERLEGLNASPTYRLYSILEGLNSERALALELEVALPRTHRLEPHESERPHEPHEALALYSSERTHRLEILEASPLYSTYRASPLESERASNLEHISGLILEALASERGLYGLYALAPRLESERLYSGLVALGLYGLALAVALALALYSARGPHEHISLEPRGLYILEARGGLNGLYTYRGLYLETHRGLTHRTHRSERALAILELEILETHRPRGLGLYASPASPLYSPRGLYALAVALGLYLYSVALVALPRPHEPHEGLALALYSVALVALASPLEASPTHRGLYLYSTHRLEGLYVALASNGLNARGGLYGLLECYSVALARGGLYPRMETILEMETSERGLYTYRVALASNASNPRGLALATHRASNALALEILEASPLYSASPGLYTRPLEHISSERGLYASPILEALATYRTRPASPGLASPGLHISPHEPHEILEVALASPARGLELYSSERLEILELYSTYRLYSGLYTYRGLNVALALAPRALAGLLEGLSERILELELEGLNHISPRASNILEPHEASPALAGLYVALALAGLYLEPRASPASPASPALAGLYGLLEPRALAALAVALVALVALLEGLHISGLYLYSTHRMETTHRGLLYSGLILEVALASPTYRVALALASERGLNVALTHRTHRALALYSLYSLEARGGLYGLYVALVALPHEVALASPGLVALPRLYSGLYLETHRGLYLYSLEASPALAARGLYSILEARGGLILELEILELYSALALYSLYSGLYGLYLYSSERLYSLE

SEQ ID NO: 3 - DNA, 5170 bp, Aequorea victoria
  misc_feature (1)..(5170): n is a, c, t, or g.
aagcttcaaattaagtcagctccttaaatgaaagataataaagtgtagttcaagaactatatgaatgatgtgttttcagataaccaaaatggggaaaaacatgctaaagtcagcatatttttggaaaattgatgacgtcatcatgacgtcgttttgatgacaaaacttattataagcgaattcttatatttttacaggataacaaagatgagtaaaggagaagaacttttcactggagttgtcccaattcttgttgaattagatggtgatgttaatgggcacaaattctctgtcagtggagagggtgaaggtgatgcaacatacggaaaacttacccttaaatttatttgcactactggaaagctacctgttccatggccaacacttgtcactactttctcttatggtgttcagtaagtgcattttatactcttttaatatcagtgttaagaaaatcaagtgtcttgctattttttcgattattggtgcaattctagtcaaattattgcgtttttttacccaaaatgttaatgtaaaactgaaatttggcacacttgcgcaaatatatacagggtattttgaaaaaattaaacaggatgataaaagttgcacagaaacttatctcaagatttacccgcagaaagatgcttnaaaaattgatatttgacagagcaaaacctgagattcacgtcttttagttgtttgacttgaaattttggtgacaggtaggtatcatgaaaaacaaacaaaacgtaaaaatatcacgtgattaaagtgtatcttacagaccagaaacagttttattaacttctattattctattttgcaatatacacattgtatcaatttcttgagttactcgaagtaataccgacctatcatcagaatttcaagtcaacacaacattatatggggctgattagggaatgattttgtctcttttagatgcttttcaagatacccagatcatatgaaacagcatgactttttcaagagtgccatgcccgaaggttatgtacaggaaagaactatattttacaaagatgacgggaactacaaatcacgtgctgaagtcaagtttgaaggtgataccctcgttaatagaattgagttaaaaggtattgattttaaagaagatggaaacattcttggacacaaaatggaatacaactataactcacacaatgtatacatcatggcagacaaacaaaagaatggaatcaaagttaacttcaaaattgtatgtatacgttaagggcataaatttttgcgggcataaaatcttgcgaaatttattatcgcgaataggttacgcaaaatctataattaaaatgtatttttttctgctgattttctaaataacaactcaacccgtcatttttatatcgcaaaaataaattccgaaataatttatgctcgcaaaaatttaggcccataagtagacttttgatatctgcgtgctctgcaatgaagtaaaaatacgatattttcattgaaatacacgggttcaaagttatttgttaattcaataagcgtgcgcagaaattaaaggacgtataaagatacgaacacatcaaaccattcatgcgtaaataatgttctatttttaaaattcaccaaagcttaaatattcttaagaattattcatgtgccatgggagcaacaatatagttatggacaaaaatttctgagttcacttttatttctgcgcgcccgcatcaaagttcaaacaactgtgaacccgagttttttccagcttgcaattttaataagagacaaaaagcaaattgcagttcaagaaaatcgagatattgccagatgtaaacatttaataagagacaaaaagttcataagcgttctaaagaacagcaacaaaataataattagaattaaacgagttctcaaacaaaataaaaactgaagtcaaagagtcagtaaggaatttagttaacgatgctttataatcaaagttttaattccagttcatgtatgcaattaacaataagatcttggagaattgaatatgtttcgaaattttataaattcggatttaatttctaaagttgtgtatcaaaaatagttcaaactattttcatgaaaagatgataaattacggtaataagtatataatataatcaattaaaattaattttaggctcaaattacagaatccacgttttttttctctagacatagcacagtgtttagatgtttgttttatttcatccatccttattacagttttcctctgaactttaatactagcgtacaatttgaataataatctgaaatgattcaacttttcagagacacaacattgaagatggaagcgttcaactagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctgggattacacatggcatggatgaactatacaaataaatgtccagacttccaattgacactaaagtgtccgaacaattactaaaatctcagggttcctggttaaattcaggctgagatattatttatatatttatagattcattaaaattttatgaataatttattgatgttattaataggggttattttcttattaaataggctactggagtgcattcctaattctatattaattacaatttgatttgacttgctcagaatcccgcttcattgcttttccacttgcattatccttatttagtattaatttgtattttggtttggctacattgagtgcaaaaaacctaattttcggacgaattttcgaacgaatttttttgacggaattttcttcattctatttactcctctagctaaattattttacctttttgttaatttggttaaattattctctgagccgatgattgagaaattaatggattaaaagtgagtaccttacatgttgtcaacttgtaacgaatggaaaaagaaattacgtttcaagagtttgaaaggtaatacagttacagttaaccgcagaaaaattgcatgatgattgataaattcgatttttgttatcctaaaattttccaaacgtcagtggccgacgactttatcagggacttctaaaagtgaaaaataatcaggtgcggatttcgaaggcgcaaaactataggaagagagcgaaatgtcattaaattatcatattctattaactgatgacaatagatgatgaaaagtttatgattattcactctcctcctgtaattatgcgacccttctagattcacgcctgaaagtatagctacctgggatgaagtactagtctgaggactcttcacctaaaaattaaattcttataagagtaaacaagaaacttagcagttacaaacgggagagcgatgagaaacaaaaacaattacgttgccactatgaatatcgatgttcaatcaattttgttccttacttataagaacgagatcgtcttaacttaaaatagtaaaatgttatcaagataatagcaattttttaccgacacagcgaagactcactactgaaatgatcagttttaatcaggcaaataatccgtggcacataatagtgaccgaaaataattaatcggcattaagactaccgaaataataatgttttttctactgcgtatacgcgtgagaaattttcaataagctcatcatcttcagcatagttatacttttatgtaaagtatcaattccgacataaaataacggcttattatcgaaataatagcgttttctctactccatgcgcgtcaaaagttctctctaggctcatcatcttcagcataattataatttttgtaaagtaccagttccggtcgaaaataatgactaattaccgaaattatagtgtttttctattgccatgcgcgtgaaaaattttgattgaatcatcatcttcagcataggcataattctttgtaaaatatcgattccgacataaaataatggcctattaccgaaataatcgcgtttttcctactgcgcatgcgcgtcaaaaattatatttttattcatcatcttcagcataattatatttttttgtaaagtaccagttccggtagaaaataatgacttgttactgaaataatagcgtttttctattgcgcatgcgctataaaaattaaagtaacgtcatcatattcagcatggtattgaaattttcaaatttaattaacctattgaacaagaatgtacacttgcatcaaaataggtgaaattcgccaatatcgctaaatgtgacgcgcgggagcaatactacgcatgtagcttcaggtaaagcatgtagaaactcggaggagtaggagtccaccgtcgaaactaaaacgggatacactacgctatggccttcgctctcccgtaaaaagggactaacaatacgacctaattgaaatactaaaaaaaacaagagaaatttaacccctttgttaacacttttcaaaagtgggattttttagccaaccatctggtatatatggttgctcattttattattatctctttctttattgttggtacaacgtagtcaaaatacaaattaggttaataaaaagcaacattataatgtataaaatctaattgtgtctaattaccgacaaattttacaggaacagttttcaccagaccgagtcttaattttagttttaaaagaaattatgtttctactgttctgacaatctgaagacaattagttctagtgtaacaatgctctgaattgaatatattcagcaatattttgtttgtaagaattggatgaatgtacgaaccttcagcagatttataccaagtgttagatttaacaagatttgcaagctgatgagtttcgagaaaattcaacatatctggatttgagggtggaacattaaaatctcctaagataataattctatcataattagaatataaattatcaatgatgtcatttaagtgatctagaaaaatattgatagtaacagttggatgtttgtatatagaaatagtaagccatctatttttcccaaatgcgagttcaaaaaccaaaattggattccttcaaagaaaaaagacattaagaaacttgatggaatcccttctcgactgtaaacaagcagtctctgggatcc

SEQ ID NO: 4 - protein, 670 aa, Aequorea Victoria
METSERLYSGLYGLGLLEPHETHRGLYVALVALPRILELEVALGLLEASPGLYASPVALASNGLYHISLYSPHESERVALSERGLYGLGLYGLGLYASPALATHRTYRGLYLYSLETHRLELYSPHEILECYSTHRTHRGLYLYSLEPRVALPRTRPPRTHRLEVALTHRTHRPHESERTYRGLYVALGLNCYSPHESERARGTYRPRASPHISMETLYSGLNHISASPPHEPHELYSSERALAMETPRGLGLYTYRVALGLNGLARGTHRILEPHETYRLYSASPASPGLYASNTYRLYSSERARGALAGLVALLYSPHEGLGLYASPTHRLEVALASNARGILEGLLELYSGLYILEASPPHELYSGLASPGLYASNILELEGLYHISLYSMETGLTYRASNTYRASNSERHISASNVALTYRILEMETALAASPLYSGLNLYSASNGLYILELYSVALASNPHELYSILEARGHISASNILEGLASPGLYSERVALGLNLEALAASPHISTYRGLNGLNASNTHRPRILEGLYASPGLYPRVALLELEPRASPASNHISTYRLESERTHRGLNSERALALESERLYSASPPRASNGLLYSARGASPHISMETILELELEGLPHEVALTHRALAALAGLYILETHRHISGLYMETASPGLLETYRLYS

SEQ ID NO: 5 - DNA, 855 bp, Clostridium difficile
tcgaagtgggcaagttgaaaaattcacaaaaatgtggtataatatctttgcttattagagcgataaacttgaatttgagagggaacttagatggtatttgaaaaaattgataaaaatagttggaacagaaaagagtattttgaccactactttgcaagtgtaccttgtacatacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaaattataaagaaggataacaaaattatacttcctttggcaattcaagttcatcacgcagtatgtgacggatttcacatttgccgttttgtaaacgaattgcaggaattgataatagttactcaggtttgtctgtaactaaaacaagtatttaagcaaaaacatgtagaaatacggtcttttttgttaccctaaaatctacaattttatacataaccacaggttagtacaaagaccttgtgtttctttttgaaaggcttaaaa

SEQ ID NO: 6 - protein, 602 aa, Clostridium difficile
METVALPHEGLLYSILEASPLYSASNSERTRPASNARGLYSGLTYRPHEASPHISTYRPHEALASERVALPRCYSTHRTYRSERMETTHRVALLYSVALASPILETHRGLNILELYSGLLYSGLYMETLYSLETYRPRALAMETLETYRTYRILEALAMETILEVALASNARGHISSERGLPHEARGTHRALAILEASNGLNASPGLYGLLEGLYILETYRASPGLMETILEPRSERTYRTHRILEPHEHISASNASPTHRGLTHRPHESERSERLETRPTHRGLCYSLYSSERASPPHELYSSERPHELEALAASPTYRGLSERASPTHRGLNARGTYRGLYASNASNHISARGMETGLGLYLYSPRASNALAPRGLASNILEPHEASNVALSERMETILEPRTRPSERTHRPHEASPGLYPHEASNLEASNLEGLNLYSGLYTYRASPTYRLEILEPRILEPHETHRMETGLYLYSILEILELYSLYSASPASNLYSILEILELEPRLEALAILEGLNVALHISHISALAVALCYSASPGLYPHEHISILECYSARGPHEVALASNGLLEGLNGLLEILEILEVALTHRGLNVALCYSLE